(19) United States
(12) Patent Application Publication
(10) Pub. No.: US 2006/0041186 A1
(43) Pub. Date: Feb. 23, 2006

(54) Continuous flow single sheath for endoscope

(76) Inventor: Thierry G. Vancaillie, Castlecrag (AU)

Correspondence Address: JAMES EPPA HITE, III, 2318 LOUIS ROAD, PALO ALTO, CA, 94303-3635, US

(21) Appl. No.: 11/205,508

(22) Filed: Aug. 16, 2005

Related U.S. Application Data
(60) Provisional application No. 60/602,741, filed on Aug. 17, 2004.

(51) Int. Cl. A61B 1/00

(52) U.S. Cl. 600/128; 600/130; 600/153

(57) ABSTRACT

An endoscopy sheath has an inside surface with longitudinally extending, inwardly projecting ridges which will surround a telescope. Contact between the ridges and the telescope creates compartments or channels within the sheath, which carry a distention medium.

This application claims the benefit of U.S. Provisional Application No. 60/602,741, filed Aug. 17, 2004.

### BACKGROUND OF THE INVENTION

1. Field of the Invention

The present invention relates generally to endoscopy, and more particularly to hysteroscope sheaths with improved flow characteristics.

2. Discussion of Prior Art

Endoscope sheaths or tubes having a visual channel for optics, possibly an instrument channel, and inflow and outflow channels for irrigating or other liquids are well-known. Diagnostic applications include Dysfunctional Uterine Bleeding (DUB) and Post-Menopausal Bleeding (PMB). Interventional (operative) applications include polypectomy, sterilization, synechiolysis, and foreign object removal. On the one hand, it is desirable to minimize the diameter of the sheath to reduce the invasive effect of its use in patients. On the other hand minimizing the diameter also reduces the capacity of the sheath to deliver and remove liquids. FIG. 1 is a cross-section of a conventional concentric-channel sheath for a diagnostic probe. Concentric sheaths cause a significant resistance to flow due to the large contact surface between the sheath and the fluid.

There remains therefore a need for an endoscopic sheath having an improved capacity for transporting liquids, while remaining of a small diameter.

### SUMMARY OF THE INVENTION

The present invention enables transporting distention medium efficiently through a single sheath surrounding a telescope used for endoscopy in bodily cavities. The invention also provides an automatic flow of medium, while maintaining distention of the cavity to be examined. This is achieved by having a higher hydraulic diameter for the inflow channel as opposed to the outflow channel. This implies that at any given time less fluid will be running out of than into the cavity. The retained fluid distends the cavity to be examined until resistance to distention is encountered, which then reduces the inflow and creates an equilibrium between flows. This automatic outflow set-up reduces the need for complicated pump systems and simplifies routine diagnostic procedures.

A preferred embodiment of a single sheath continuous flow system for endoscopy according to the invention includes a single oval to round tubular structure which conforms to the outside of a telescope, with or without an instrument alongside it. The sheath has internal ridges which, when touching the outside of the telescope, create compartments of unequal hydraulic diameter between the endoscope and the sheath. The inflow area is larger than the outflow area. Under certain circumstances for a passive outflow, a slit is formed along the sheath at a distance from the tip which is introduced in the bodily cavity. This slit is located along the compartment which carries the outflow fluid. This allows a constant leakage of fluid, thus creating a continuous flow of medium through the system of endoscope and cavity. The continuous flow is an advantage as it allows blood and other bodily fluids which obscure the view to be eliminated.

Among the advantages of the invention are that: reducing the number of sheaths from two to one potentially reduces their diameter; a single sheath system is easier to assemble; unequal compartments allow for automatic distention of bodily cavity; and the structure of the channels increases the hydraulic diameter and thus rheologic properties of the system. These and other advantages of the present invention will become apparent to those skilled in the art upon reading the following detailed description of the preferred embodiments as shown in the several figures of the drawing.

### BRIEF DESCRIPTION OF THE DRAWING

FIG. 1 is a cross-section of a conventional concentric-channel diagnostic probe;

FIG. 2 is a cross-section showing example dimensions of an off-center-channel diagnostic probe according to the invention having ridges on the inside of a single sheath to create two unequal compartments when in contact with a telescope;

FIGS. 3A, 3B and 3C are cross-sections of a diagnostic endoscope sheath using one ridge to create two fluid channels according to the invention;

FIGS. 4A, 4B and 4C are cross-sections of an operative endoscope sheath according to the invention using two ridges to create three channels, the third for the introduction of an instrument;

FIG. 5 is a side view of a Storz® endoscope of the type to be inserted through a sheath;

FIG. 6 shows a side view of a sheath having a passive outflow slit in the sheath along the outflow channel;

FIG. 7 shows a side view of a sheath having an active outflow which can be connected to suction; and

FIG. 8 shows a side view of a sheath having an active outflow and an instrument channel.

### DETAILED DESCRIPTION OF THE INVENTION

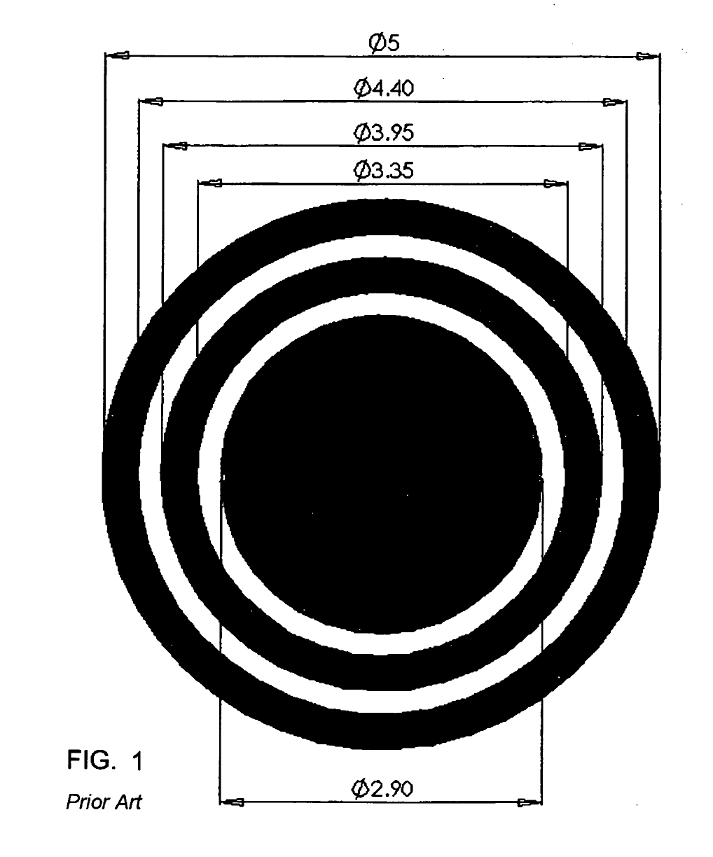

The present invention uses radial ridges along the inside of a sheath surrounding the telescope. Contact between the ridges and the telescope creates compartments or channels within the sheath, which carry the distention medium. FIG. 2 is a cross-section showing example dimensions of an off-center-channel diagnostic probe according to the invention. The ridges mounted on the inside of the single sheath create two unequal compartments when in contact with the telescope. These compartments are used as channels for in- and out-flow of distention medium. Comparing FIGS. 1 and 2, it is evident that the invention's use of transverse ‘ribs’ increases the area available for inflow and outflow of distention medium.

The replacement of conventional co-axial tubes system of irrigation by the present invention using a longitudinal ridge segmented system offers the following advantages:

First, less volume of sheath wall material is used to separate channels, preserving more cross-sectional area for fluid flow. More area means less friction between the fluids and the channel wall surfaces.

Second, the cross-sectional shape of the flow channel is changed to consolidate its area and shorten its perimeter, again resulting in less friction. Less contact between surface and liquid means less friction and therefore greater flow.

The increase and consolidation of flow area combine to produce a significantly better flow characteristic for the segmented system. In calculating the so-called hydraulic diameter, the segmented system allows a diameter which is three times that of the co-axial system. This translates into a flow which is 3×3=9 times higher, as flow is relative to the square of the diameter. Hence, re-arranging the available space around the optic increases flow characteristics by a full order of magnitude.

Using as an example the dimensions of the FIG. 1 conventional concentric-channel probe:

Inflow (the Duct Nearest the Optic):

Cross-sectional area=2.21 mmˆ2, wetted perimeter=19.63 mm
Hydraulic diameter (Dh)=0.45 mm
   Outflow:
Cross-sectional area=2.95 mmˆ2, wetted perimeter=26.23 mm
Hydraulic diameter (Dh)=0.45 mm
   Using as an example the dimensions of the FIG. 2 off-center channel probe according to the invention:
   Inflow (the Larger of the Two Spaces):
Cross-sectional area=4.96 mmˆ2, wetted perimeter=11.07 mm
Hydraulic diameter (Dh)=1.79 mm
   Outflow:
Cross-sectional area=2.86 mmˆ2, wetted perimeter=8.18 mm
Hydraulic diameter (Dh)=1.39 mm

As shown in FIG. 2, the channels form a sharp angle with the optic, which in this design is reduced by creating an inner ridge on the sheath which hugs the optic. This angle is critical to flow. Narrow angles create turbulence in the flow of distention medium, depending on a number of issues relating to the medium which can not be anticipated when constructing a sheath for the scope. The bottom of the opening between the sheath's inner wall surface and the outer surface of the optic member could be a curve tangent to both surfaces. Based upon current commonly-used distention media, an optimum shape of the ridges on the inside of the sheath can be calculated to blunt these sharp angles.

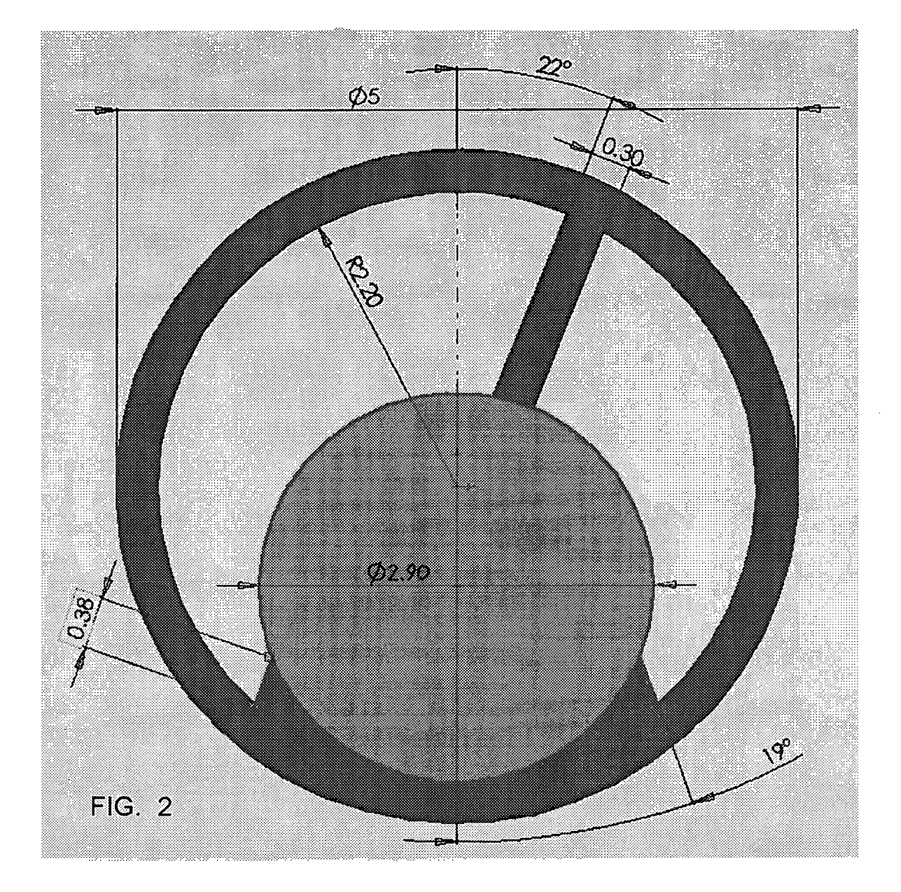

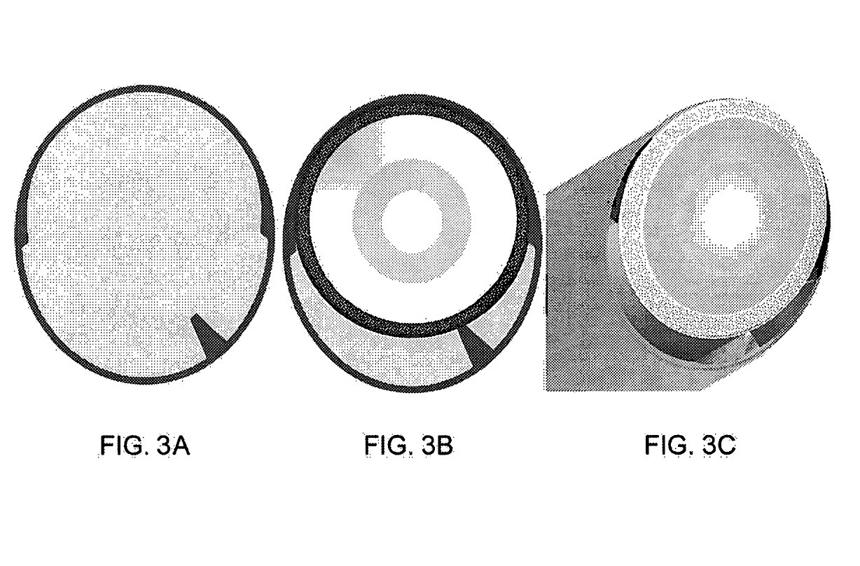

FIGS. 3A, 3B and 3C are cross-sections of a diagnostic endoscope sheath according to the invention using one ridge to create two channels. The sharp edges with the endoscope are blunted to reduce troublesome turbulence. FIG. 3A shows the sheath without a telescope element installed. FIG. 3B shows the sheath with a telescope element installed. FIG. 3C is a cutaway perspective view of a sheath holding a telescope element.

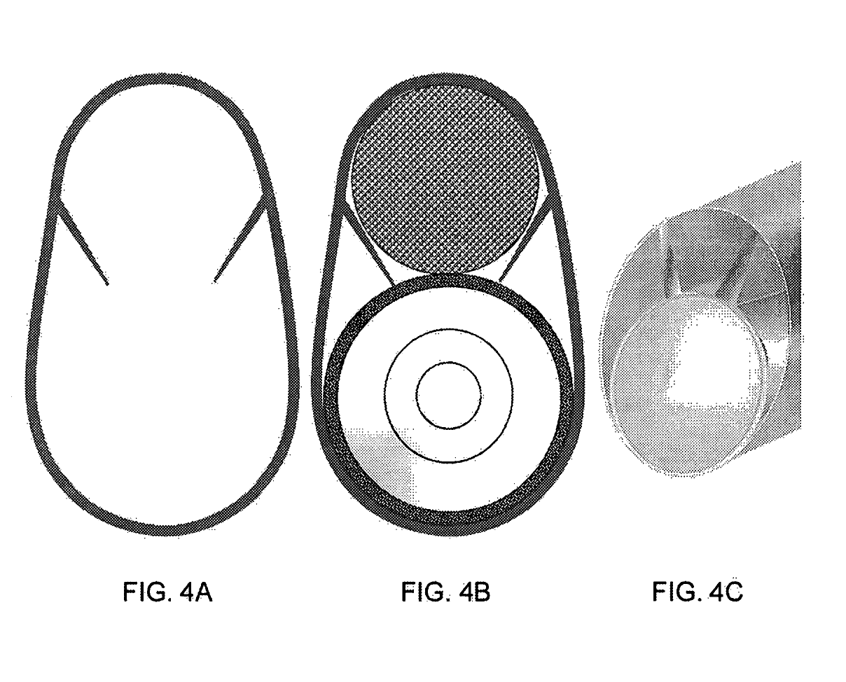

FIGS. 4A, 4B and 4C are cross-sections of an operative endoscope sheath according to the invention, in an alternate embodiment using two ridges to create three channels. FIG. 4A shows the sheath empty. In addition to the two fluid channels, a third channel allows for the introduction of an instrument. FIG. 4B shows the sheath with a telescope element and an operative element both installed. FIG. 4C is a cutaway perspective view of a sheath holding a telescope element and an operative element.

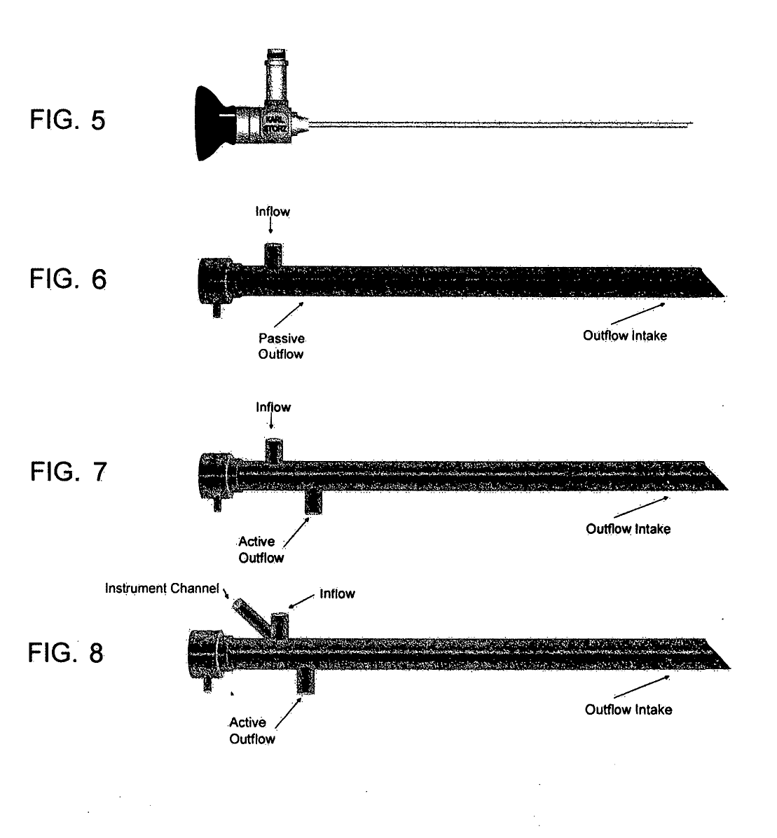

FIG. 5 is a side view of a Storz® endoscope of the type to be inserted through the diagnostic element channel of sheaths according to the invention. The segmented system having two components (sheath and telescope) rather than three makes the system of the invention friendly to assemble in an operating theatre.

FIG. 6 is a side view of a sheath having a passive outflow slit in the sheath along the outflow channel at a distance from the tip of the endoscope. This allows a small volume of fluid to escape, thus creating a continuous flow.

FIG. 7 is a side view of a sheath having an active outflow which can be connected to a source of suction to increase the amount of fluid flowing through the system.

FIG. 8 is a side view of a sheath having an active outflow as well as an instrument channel.

While the present invention is described in terms of several preferred embodiments, it will be appreciated by those skilled in the art that these embodiments may be modified without departing from the essence of the invention. It is therefore intended that the following claims be interpreted as covering any modifications falling within the true spirit and scope of the invention.

I claim:

1. An endoscopy sheath comprising:
a hollow cylinder including a cylinder wall having an interior surface with at least one longitudinal rib projecting inwardly to fit against a tubular member inserted lengthwise through the cylinder so that the interior surface, the rib and the tubular member together form separate channels on either side of the rib.

2. The sheath of claim 1 wherein an arc section of the interior surface forms a cradle portion having a shorter radius than that of other arcs around the interior surface and the inserted tubular member seats in the cradle portion, so that the ends of the arc section reduce the acuteness of the angle between the interior surface and the tubular member, whereby turbulence of fluid flow through the channels is reduced from what it would be without the cradle portion.

3. The sheath of claim 1 wherein said cylinder has a non-circular cross-section.

4. The sheath of claim 1 wherein said cylinder wall, at a distance from a discharge end of the cylinder, has a passive outflow slit along an outflow channel which allows a small volume of fluid to escape, thus creating an automatic flow.
